(19) United States
(12) Patent Application Publication
(10) Pub. No.: US 2018/0209050 A1
(43) Pub. Date: Jul. 26, 2018

(54) HYDROGEN GENERATION APPARATUS

(71) Applicant: Seung Ho TAK, Seoul (KR)

(72) Inventor: Seung Ho TAK, Seoul (KR)

(21) Appl. No.: 15/746,562

(22) PCT Filed: Jul. 20, 2016
(86) PCT No.: PCT/KR2016/007915

§ 371 Date: Jan. 22, 2018

(30) Foreign Application Priority Data
Jul. 21, 2015 (KR) 10-2015-0103257

(51) Int. Cl. C25B 1/04; C25B 9/06; C25B 11/03; C25B 11/04; C25B 15/02; A61M 15/06; A61M 15/02

(52) U.S. Cl. CPC C25B 1/04; C25B 9/06; C25B 11/035; C25B 11/0447; A61M 2205/3331; A61M 15/06; A61M 15/02; A61M 2205/587; A61M 2205/3317; C25B 15/02

(57) ABSTRACT

In a hydrogen generation apparatus, water is electrolyzed by an electrolysis electrode part, and a porous ceramic catalyst may be combined with the oxygen generated at the time of electrolysis to prevent the generation of ozone. In addition, when the resistance value of water is measured, the change in resistance according to the kind of electrolyte dissolved in water and the concentration of the corresponding electrolyte is measured, and then, the applied voltage is varied depending on the measured resistance value, so that the water is electrolyzed in the optimal quantity according to the ingredient dissolved in the water, thereby preventing the generation of components harmful to the human body.

### TECHNICAL FIELD

The present invention relates to a hydrogen and hydrogen water generation technology using an electrode, and more particularly, to an electrolytic hydrogen water generation technology in which hydrogen gas is generated by applying a voltage to an electrode plate in water.

### BACKGROUND ART

Active oxygen, which is also called as harmful oxygen, is generated by oxygen being excessively generated due to chemicals, ultraviolet rays, stress, blood circulation disorders, and the like, and the active oxygen performs oxidation in the human body and damages cell membranes, deoxyribonucleic acid (DNA), and cells. In addition, the active oxygen oxidizes various amino acids in the body and causes functional degradation of proteins. The effect of the active oxygen may cause a decrease in a physiological function, and various diseases and aging. It is known that about 90% of modern diseases are related to the active oxygen.

Hydrogen can be used to remove the active oxygen. The hydrogen is chemically combined with activated oxygen to obtain an antioxidant effect. Accordingly, when a person drinks hydrogen water in which hydrogen is dissolved in water or breathes hydrogen itself or the skin comes into contact with the hydrogen water, the person can obtain an antioxidant effect. Recently, it has become popular to manufacture and drink hydrogen water.

In “Apparatus for Manufacturing Hydrogen Water” (KR Patent Laid-open No. 10-2013-0073831 (Jul. 3, 2013)), an apparatus configured to produce hydrogen water by electrolyzing water is disclosed.

### DISCLOSURE

### Technical Problem

An electrolyte has to be contained in water to generate hydrogen by electrolyzing the water. In a case of distilled water, since an electrolyte is not present in the water, current does not flow in the water, and thus the distilled water cannot be electrolyzed. However, when water is electrolyzed, since an electrolyte causes an electrochemical reaction according to the concentration of the electrolyte dissolved in the water, the electrolyte itself may be electrolyzed. Specifically, the electrolyte may be reduced or the water may be reduced to generate hydrogen according to the degree that an electrolyte is dissolved in water and remains as ions, that is, reactivity.

In the case of K⁺, Ca₂⁺, Mg₂⁺, Al⁺, or the like among positive ions of an electrolyte, since reactivity thereof is high, the K⁺, Ca₂⁺, Mg₂⁺, Al⁺, or the like is reduced to generate H₂, and in the case of Au, Pt, Ag, Hg, Cu, or the like, since reactivity thereof is lower than that of the hydrogen ions, the electrolyte is reduced. Since ions such as F, NO₃, CO₃, or SO₄ among negative ions of an electrolyte have high reactivity, a tendency to remain as ions is strong, and thus water is oxidized to generate O₂, and since ions such as Cl have reactivity lower than that of oxygen ions, an electrolyte is oxidized. Therefore, an electrolyte, which can be used to electrolyze water, contains positive or negative ions having high reactivity, and distilled water may not be electrolyzed because positive and negative ions having low reactivity are contained.

In the case of salt water, electrolysis is divided into aqueous NaCl solution electrolysis and NaCl molten electrolysis on the basis of concentration of salt dissolved in water, and electrolysis chemical equations of a salt molten state in which pure NaCl is melted in water are as follows.

Positive Electrode: 2Cl⁻→Cl₂(g)+2e⁻

Negative electrode: 2Na⁺+2e⁻→2Na(s)

Cl⁻ (ion state) discharges electrons and attains a gaseous state, and Na⁺ (ion state) obtains electrons and attains a solid state. When Na precipitates in a NaCl solution, the Na, which is a group 1 element, is explosively combined with water, however, when the Na is not precipitated therein, hydrogen gas is generated.

In a case in which salinity is high according to the concentration of salt dissolved in water, salt water is electrolyzed (NaCl+H₂O→Na⁺+Cl⁻+H₂),

That is, when salt water is electrolyzed, chemical equations are as follows.

Positive electrode (oxidation): 2Cl⁻(Ag)→Cl₂(g)+2e⁻

Negative electrode (reduction): 2H₂O(l)+2e⁻→H₂(g)+2OH⁻

Total reaction: Cl⁻(Ag)30 H₂O(l)→Cl₂(g)+H₂(g)+2OH⁻(Ag)

That is, chlorine gas and hydrogen gas are generated and a solution becomes NaOH alkali.

Since variously commercialized apparatuses configured to electrolyze water to generate hydrogen cause various chemical reactions on the basis of an electrolyte dissolved in water, the apparatuses may not achieve original objects thereof according to a type of water, that is, the concentration of salt in water to be electrolyzed.

### Technical Solution

One aspect of the present invention provides a hydrogen generation apparatus including an electrolysis electrode part which electrolyzes the water and in which a porous ceramic catalyst combined with oxygen generated during the electrolysis to prevent the generation of ozone is located, a resistance measuring part configured to measure a resistance of the water and measure a change in the resistance based on a type of an electrolyte dissolved in the water and a concentration of the corresponding electrolyte, a power supply part configured to apply a voltage isolated from a power device to electrolysis electrodes, a control part configured to control the power supply part to change and apply the voltage on the basis of the resistance measured by the resistance measuring part, and a relay part controlled to apply the voltage to the electrolysis electrodes while changing a direction of current at a predetermined time interval.

In one aspect, the hydrogen generation apparatus may further include a light-emitting diode configured to flicker or light up in a combination of red, blue, and a green to inform of operating and stopping of electrolysis. In addition, the control part may control the relay part to stop the supply of power to stop electrolysis in a case in which the resistance measuring part measures a resistance of the water in which a salinity is 10% or more and control the light-emitting diode to inform of the stopping of electrolysis.

In one aspect, the control part may control the relay part to decrease the applied voltage in a case in which the resistance measuring part measures a resistance of the water in which a salinity is equal to or higher than 4% and lower than 10%, and to electrolyze the water for an added voltage application time period corresponding to the lowered voltage.

In one aspect, the power supply part and the relay part may be controlled to electrolyze the water at a maximum voltage for a predetermined time period in a case in which the resistance measuring part measures a resistance of water in which a salinity is lower than 3%.

In one aspect, the resistance measuring part may measure a change amount of the resistance based on an amount of water, and the control part may control the relay part to supply power on the basis of the change amount of the measured resistance to change a time for which water is electrolyzed.

In one aspect, the control part may control the power supply part to apply a voltage lower than an initial voltage isolated from a power supply voltage in a case in which a resistance between both ends of the electrolysis electrodes is not measured and the initial voltage is applied to the both ends of the electrode and excessive current flows at the both ends of the electrodes.

In one aspect, the hydrogen generation apparatus may further include a gyro sensor configured to detect an inclination of a water bottle, wherein the control part may control the power supply part to stop the supply of power in a case in which an inclination of the water bottle detected by the gyro sensor is greater than a predetermined angle.

In one aspect, the hydrogen generation apparatus may further include a water level detecting electrode configured to detect contact with water, wherein the control part may control the power supply part to stop the supply of power in a case in which the water level detecting electrode detects contact with water.

In one aspect, the hydrogen generation apparatus may further include a pressure sensor configured to detect a pressure of the hydrogen generated by the water being electrolyzed, wherein the control part may control the power supply part to stop the supply of power in a case in which a pressure detected by the pressure sensor is higher than a predetermined pressure.

In one aspect, the hydrogen generation apparatus may further include a use management part configured to manage use information of a user based on control content of the control part, wherein the use management part may further include a storage part which stores the use information and in which modification is impossible after the use information is stored.

In one aspect, the hydrogen generation apparatus may further include a communication part connected to an apparatus use expert system and configured to transmit the use information to the apparatus use expert system, or receive a guide about the use information from the apparatus use expert system.

In one aspect, the hydrogen generation apparatus may include a waterproof space including a first magnetically-sensitive power isolation switch operated by a magnetic force without physical contact with a magnet to adjust power of the hydrogen generation apparatus to be turned on and off.

In one aspect, hydrogen generation apparatus may further include a waterproof space separated from the electrolysis electrode part, and sealed to prevent contact with water.

In one aspect, the waterproof space may further include a second magnetically-sensitive power isolation switch adjusted by a magnetic force without physical contact with a magnet, and an exposed boosting charge part including an exposed boosting charge electrode configured to protrude outward from the waterproof space, and connected to the magnetically-sensitive power isolation switch to charge a secondary battery in a case in which the exposed boosting charge electrode comes into contact with a magnet.

In one aspect, the hydrogen generation apparatus may include a main body including the waterproof space, wherein the corresponding main body may include a water bottle coupling portion including a female thread formed at a lower end of the main body to couple the main body to a water bottle, a rotary switch having a ring structure and which operates by rotating the first magnetically-sensitive power isolation switch, an electrolysis electrode part formed in a cylindrical shape and including the electrolysis electrode part, the power supply part, and a protruding part formed at an upper end of the electrolysis electrode part, and a fixing groove into which the protruding part is inserted to couple the electrolysis electrode part to the main body, and the electrolysis electrode part may protrude downward from the lower end of the main body after the protruding part is coupled to the fixing groove and enter the water bottle when the main body is coupled to the water bottle.

In one aspect, the main body may further include a vertical path through which water passes, and a cap coupling portion including a male thread formed at an upper end of the cap coupling portion to be coupled to a cover configured to open and close the path.

In one aspect, the main body or the cover further include a nozzle connected to a hose of a hydrogen respirator.

Another aspect of the present invention provides a hydrogen generation apparatus having an electronic cigarette form, including a power supply part including a charge circuit, a secondary battery, and an isolated DC-DC converter, a hydrogen generation part coupled to the power supply part at one side and configured to be separable from the power supply part, and an inhalation part coupled to the hydrogen generation part at the other side opposite the one side, wherein a porous ceramic catalyst configured to be combined with oxygen generated when water is electrolyzed to prevent the generation of ozone is located in the hydrogen generation part, and a water bottle includes a liquid absorbent in which parts of electrolysis electrodes are buried at one side at which the electrolysis electrodes in a cylindrical shape having a vertical path are coupled to an inhalation part.

In one aspect, the inhalation part may include an inhalation part through which a user inhales hydrogen, a micro hole through which external air to be mixed with the hydrogen enters when the user inhales the hydrogen using the inhalation part, and an adjustor configured to open and close the micro hole by rotating.

### Advantageous Effects

According to the embodiment of the present invention, in a hydrogen generation apparatus, since a voltage is adjusted on the basis of a component dissolved in water to appropriately electrolyze, a level of reliance on water is improved. In addition, since the hydrogen generation apparatus is coupled to a water bottle generally used for daily life, the accessibility of hydrogen water to the public is increased.

### DESCRIPTION OF DRAWINGS

FIG. 1 is a block diagram of an internal circuit configuration of a hydrogen generation apparatus.

FIG. 2 is a graph showing a voltage and current which are applied and a measured resistance according to time when the hydrogen generation apparatus is operated.

FIG. 3 is a view illustrating an algorithm in which applied power is changed on the basis of a value measured by a resistance measuring part of the hydrogen generation apparatus.

FIG. 4 is a view illustrating a configuration of the hydrogen generation apparatus including a water bottle and a main body.

FIG. 5 is a view illustrating one example of the hydrogen generation apparatus including a main body couplable to a general mineral water bottle.

FIG. 6 is a view illustrating one example of the hydrogen generation apparatus including a nozzle at a cap thereof and an image in which a person breathes through a hydrogen respirator installed at the corresponding nozzle.

FIG. 7 is a view illustrating an example of an electric electrode rod.

FIG. 8 is a view illustrating a configuration of the hydrogen generation apparatus having an electronic cigarette form.

FIGS. 9, 10, 11, and 12 are views illustrating the hydrogen generation apparatus capable of being immersed in water.

FIG. 13 is an exploded view illustrating one example of the hydrogen generation apparatus configured to be immersed and used in water.

### MODES OF THE INVENTION

Above-described and additional aspects of the present invention will become further apparent through embodiments which will be described below. Hereinafter, the embodiments will be described in detail with reference to the accompanying drawings enough to be understood and reproduced by those skilled in the art.

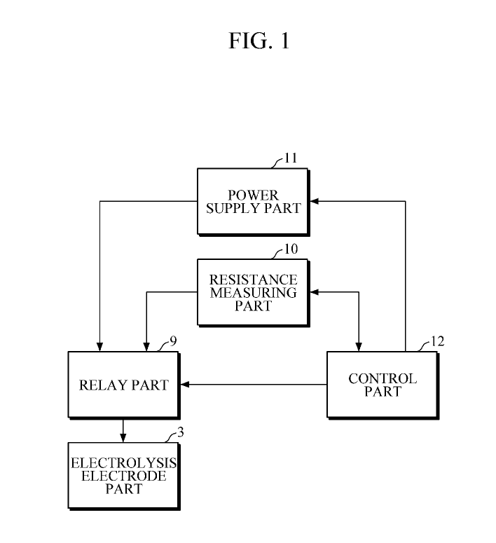

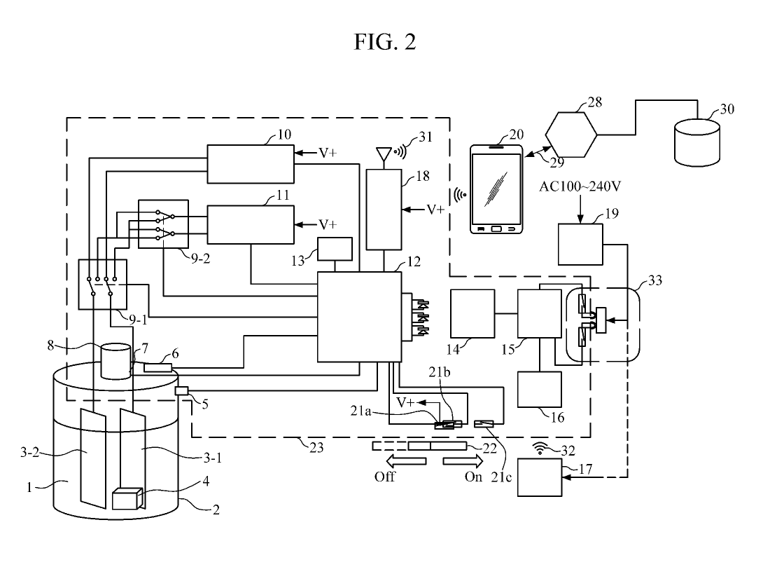

FIG. 1 is a block diagram of an internal circuit configuration of a hydrogen generation apparatus according to the present invention. FIG. 2 is a schematic block diagram of the configuration of the view of FIG. 1. As illustrated in FIG. 1, the hydrogen generation apparatus may include an electrolysis electrode part 3, a resistance measuring part 10, a power supply part 11, a control part 12, and a relay part 9.

In one embodiment, the electrolysis electrode part 3 includes a positive electrode and a negative electrode, and the corresponding electrodes are disposed to be in contact with water 1. When power is supplied to the corresponding electrodes, the water 1 in contact with the corresponding electrodes is electrolyzed. In one aspect, a porous ceramic catalyst 4 may be located in the electrolysis electrode part 3 and combined with oxygen generated when water is electrolyzed to prevent the generation of ozone. In FIG. 1, an example of the porous ceramic catalyst 4 located between electrolysis electrodes 3-1 and 3-2 is illustrated. The porous ceramic catalyst 4 is a ceramic made by synthesizing various minerals such as magnesium, iron oxide, and tourmaline. When the water 1 is electrolyzed into two hydrogen atoms and one oxygen atom, ozone may be generated, and when the corresponding catalyst 4 is disposed at the electrolysis electrode part 3, the generation of ozone may be prevented because the oxygen atoms and the ceramic catalyst 4 are combined before ozone is generated. In one embodiment, a hydrogen generation chemical equation is represented by 2H₂O+Mg→Mg(OH)₂+H₂, and since magnesium consists of two electrons in a K shell, eight electrons in an L shell, and two electrons in an M shell and two outermost electrons among the electrons are in an unstable state, the two electrons may be easily released, and thus the magnesium has reducing power. In a case in which magnesium reacts with water, one magnesium molecule reacts with two water molecules, at this moment, the magnesium is not liberated and magnesium hydroxide is generated, and in this process, some of the electrons emerging from the magnesium are used to generate hydrogen gas and the remaining electrons remain in water. The magnesium hydroxide is ionized to generate a hydroxyl group (OH⁻), at this moment, monoatomic oxygen is combined with ozone (O₃) instead of an oxygen molecule, in this case, the monoatomic oxygen combined with the ozone is changed into general oxygen, and ozone disappears. That is, the magnesium is oxidized, instead, the water is reduced to become hydrogen water, and when a person drinks the hydrogen water made by mixing the electrolysis hydrogen gas and the ceramic formed of minerals such as magnesium, iron oxide, tourmaline, and like and breathes the hydrogen gas, only a hydroxyl radical most harmful among active oxygen is selectively combined with hydrogen to become H₂O, and thus an antioxidation effect is achieved. Such antioxidant water has a minus (−) oxidation and reduction electric potential value, and its reducing power is superior, and the electric potential value ranges from −100 mV to −1989 mV.

In one aspect, the resistance measuring part 10 may measure a resistance of the water 1, and a change in the resistance based on a type of an electrolyte dissolved in the water and a concentration of the corresponding electrolyte. In one embodiment, the resistance measuring part 10 is an analog-to-digital converter. The resistance measuring part 10 measures a resistance of water for a predetermined time period in a state in which power supply is stopped. Here, the relay part 9 may stop power supply, which will be described below.

In one embodiment, the resistance measuring part 10 may measure the resistance of the water in consideration of a type and a concentration of the electrolyte dissolved in the water. For example, in a case in which a factor that mostly affects electrical resistance is a concentration of salt, that is salinity, among various electrolytes dissolved in water, the resistance measuring part 10 may measure the present resistance and measure the present salinity based on the measured resistance. Alternatively, the resistance measuring part 10 may measure a change in salinity of water based on a change in electrical resistance of the water. Here, a different electrolyte which may become a gas harmful to the human body due to a chemical reaction during electrolysis may be considered in addition to the salinity.

In one aspect, the power supply part 11 may apply a voltage isolated from a power device to electrolysis electrodes 3. In one embodiment, the power supply part 11 is a direct current (DC)-DC converter. Such a power supply part 11 is a step-up DC-DC converting circuit, in which a first circuit is completely isolated from a second circuit so that the electrodes in contact with water may be completely independent from the power device and power may be supplied to the electrodes, and supplies a voltage to the electrolysis electrode part. In addition, the power supply part 11 supplies power to the control part 12 and the like, which will be described below, and allows the hydrogen generation apparatus to operate.

In one aspect, the control part 12 controls the power supply part 11 to change a voltage on the basis of a resistance measured by the resistance measuring part 10 to apply the voltage to the electrodes. That is, since a type and a concentration of an electrolyte may be known using the measured resistance, a voltage may be changed on the basis of the type and the concentration of the corresponding electrolyte. Although it will be described below, a voltage to be applied is changed according to the measured resistance in consideration of an effect of a concentration of electrolyte on the human body according to the result of electrolysis. For example, when a resistance of an electrolyte such as an iron salt is low, an electrolysis voltage and electrolysis current are reduced and supplied to the electrodes, and in a case in which a resistance of an electrolyte is high, a maximum rated voltage and appropriate current are supplied to the electrodes. Graphs 35 and 36 in FIG. 2 show examples in which magnitudes of voltages V1 and V2 are different according to resistance.

In one aspect, the relay part 9 may be controlled to apply a voltage to the electrolysis electrode part while changing a direction of current at a predetermined time interval. In one embodiment, the relay part 9 may be a latch relay or solid state relay circuit. The relay part 9 reverses polarities of power supplied to the electrodes at a predetermined time interval to prevent a chemical migration phenomenon of the both electrodes.

In one aspect, the hydrogen generation apparatus may include a light-emitting diode 56. The light-emitting diode 56 may flicker or light up in a combination of red, blue, and green to inform of operating and stopping of electrolysis. The control part 12 may control flickering and lighting up of the light-emitting diode 56. In one embodiment, the light-emitting diode 56 may have colors other than the above-described colors.

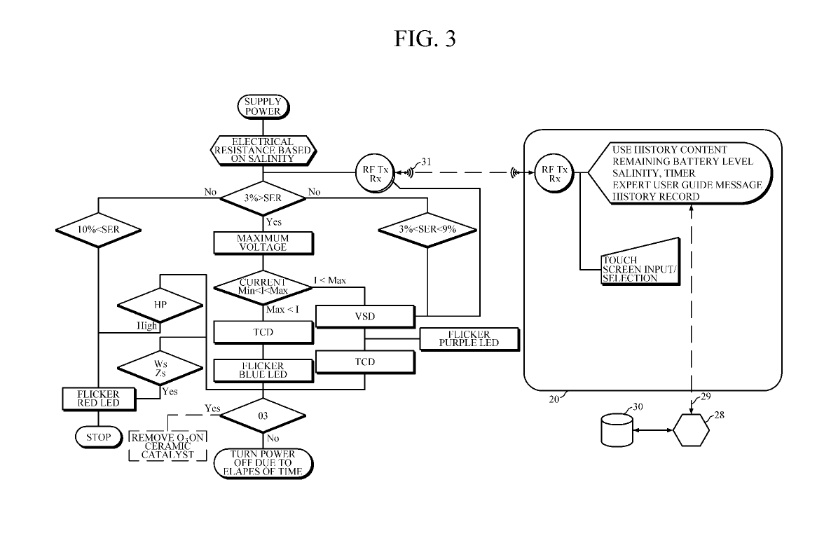

FIG. 3 is a view illustrating an algorithm of operation of the hydrogen generation apparatus based on a measured resistance. Hereinafter, the algorithm of FIG. 3 will be described with reference to the block diagram of FIG. 2.

In one aspect, the control part 12 may control the relay part 9 to stop power supply and electrolysis in a case in which the resistance measuring part 10 measures a resistance of water when a salinity thereof is 10% or more. At the same time, the control part 12 may control the light-emitting diode 56 to inform of stopping of electrolysis. This is to prevent generation of gas other than hydrogen gas because, in a case in which a salinity of water is 10% or more (for example, seawater), a content of an electrolyte is high and high current flows compared to an applied voltage and thus the current higher than an allowable current.

In addition, the control part 12 may control the light-emitting diode 56 to inform of stopping of electrolysis by flickering or lighting up the light-emitting diode 56 in red as described above.

In another aspect, the control part 12 may control the power supply part 11 to decrease a voltage to be applied in a case in which the resistance measuring part 10 measures a resistance of water in which a salinity is equal to or higher than 4% and lower than 10%. In the case in which the salinity of the water is equal to or higher than 4% and lower than 10%, since generation of gas other than hydrogen gas may be prevented by applying a lowered voltage, electrolysis is not immediately stopped like when the salinity is 10%. In addition, the control part 12 may control the relay part 9 to electrolyze water for an added voltage application time period corresponding to the lowered voltage to generate an appropriate amount of hydrogen gas. In addition, the control part 12 may control the light-emitting diode 56 to flicker or light up in green to inform a user of a present electrolysis state. In one embodiment, in a case in which current in a range corresponding to a resistance of water having a salinity, which is lower than 3%, flows when a lowered voltage is applied because a salinity is equal to or higher than 4% and lower than 10%, a blue light-emitting diode 56 may be flickered or lighted up to inform of a present electrolysis state.

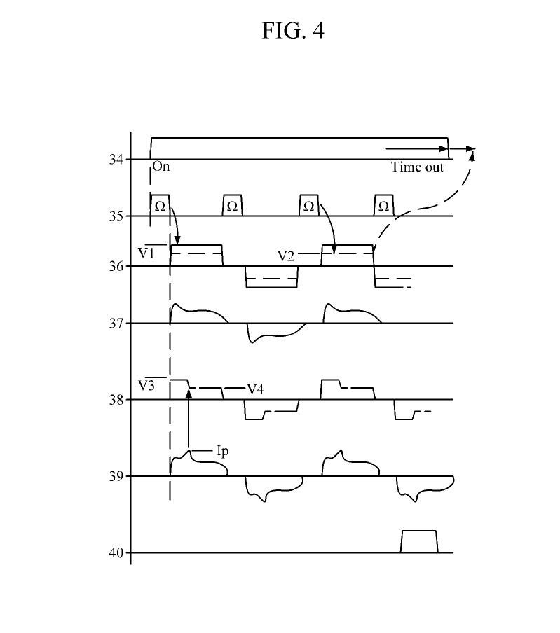

In one aspect, the control part 12 may control the power supply part 11 and the relay part 9 such that water is electrolyzed at a maximum voltage for a predetermined time period in a case in which the resistance measuring part 10 measures a resistance of water in which a salinity is lower than 3%. In this case, since a rate of salinity is low, it is difficult for current higher than an allowable current to flow even when a maximum voltage is applied. A graph 36 of FIG. 4 is a graph in which a voltage Vmax is applied for a predetermined time period. As illustrated in the algorithm of FIG. 3, when the Vmax is applied to the electrolysis electrode part 3 and current flows within an appropriate range, electrolysis is performed for a predetermined time period and the blue light-emitting diode 56 may flicker or may be lighted up to inform a user of a state of electrolysis.

In one aspect, the resistance measuring part 10 may measure a change amount of the resistance based on an amount of water. In one embodiment, the resistance measuring part 10 may measure a resistance that is minutely decreased or increased by the areas of the electrolysis electrodes in contact with water being changed according to an amount of water. The resistance measuring part 10 transmits information about the amount of the changed resistance to the control part 12 whenever a resistance minutely changed on the basis of a change in an amount of water is measured.

In one aspect, the control part 12 may control the relay part to change a time for which water is electrolyzed on the basis of a change amount in a case in which the resistance measuring part 10 measures a change in resistance based on an amount of water. As described above, when an amount of water in a container is changed and thus the areas of the electrolysis electrodes 3 in contact with the water are changed, the resistance measuring part 10 measures a resistance due to the change in area and immediately transmits the measured resistance to the control part 12, and the control part 12 controls the relay part 9. In one embodiment, when the contact area increases due to an increase in an amount of water, a resistance decreases minutely, and thus switching of the relay part 9 is controlled to decrease a time of electrolysis. Conversely, when the contact area decreases due to a decrease in an amount of water, a resistance increases minutely, and thus switching of the relay part 9 is controlled to increase a time of electrolysis.

In one aspect, the control part 12 may control a power supply part 11 to apply a voltage lower than an initial voltage isolated from a power supply voltage in a case in which a resistance between both ends of the electrolysis electrodes 3 is not measured and the initial voltage is applied to the both ends of the electrodes and excessive current flows at the both ends thereof. In a case in which, after a voltage V3 is applied to the electrolysis electrodes 3 as illustrated in a graph 38 of FIG. 4, an excessive current Ip flows as illustrated in a graph 39, the control part 12 controls the power supply part 11 to decrease the voltage to V4 and decrease current. In this control process, a resistance is not measured as shown in a graph 40 of FIG. 4, and after a voltage is controlled to decrease current, the resistance is measured.

In one aspect, the hydrogen generation apparatus may include a water bottle 2 and a gyro sensor 6. The water bottle 1 may accommodate water which will be in contact with the electrodes 3 of the hydrogen generation apparatus. The gyro sensor 6 detects an inclination of the corresponding water bottle 2, and an operational principle of the sensor will not be described because the operational principle is clear to those skilled in the art.

In one aspect, the control part 12 may control the power supply part 11 to stop the supply of power in a case in which an inclination of the water bottle measured by the gyro sensor 6 is greater than a predetermined angle. Since water may leak from the water bottle when the water bottle 2 is inclined greater than a predetermined angle, the control part 12 controls the power supply part 11 to stop the supply of power in advance.

In one aspect, the hydrogen generation apparatus may further include a water level detecting electrode 7 configured to detect whether the water level detecting electrode 7 comes into contact with water. The water level detecting electrode 7 may be disposed at an upper end of the water bottle 2 and check how much the water 1 is accommodated in the water bottle 2. The water level detecting electrode 7 may be disposed at a suitable location on the basis of a size or shape of the water bottle 2.

In one aspect, the control part 12 may control the power supply part 11 to stop the supply of power in a case in which the water level detecting electrode 7 detects contact with water. In one embodiment, when the water level detecting electrode 7 detects contact with water, water is accommodated in the water bottle up to the water level detecting electrode 7 or to a level higher than a height of the water level detecting electrode 7, that is, a remaining space in the water bottle 2 is not large. In this case, when electrolysis is continuously performed and hydrogen gas is continuously generated, internal pressure in the water bottle 2 may become excessively high. Accordingly, when the water level detecting electrode 7 detects contact with water, the detected signal is immediately transmitted to the control part 12 such that the control part 12 controls the power supply part 11.

In one aspect, the hydrogen generation apparatus may further include a pressure sensor 5. The pressure sensor 5 may detect a pressure of hydrogen generated by water electrolysis. The pressure sensor 5 detects a pressure of hydrogen gas in the water bottle and transmits a detected signal to the control part 12 such that the control part 12 controls electrolysis on the basis of the detected pressure.

In one aspect, control part 12 may control the power supply part 11 to stop electrolysis in a case in which a pressure detected by the pressure sensor 5 is higher than a predetermined pressure. In one embodiment, in a case in which the water being electrolyzed is accommodated in a small space such as a plastic bottle or a polyethylene terephthalate (PET) bottle and a pressure of hydrogen gas generated by water being electrolyzed is higher than a predetermined pressure, a safety problem such as an explosion may occur. Accordingly, when the pressure sensor 5 detects a pressure higher than the predetermined pressure, the pressure sensor 5 transmits a detected signal to the control part 12 such that the pressure does not reach a critical level, and the control part 12 controls the power supply part 11 to stop the supply of power on the basis of the detected signal.

In one aspect, the hydrogen generation apparatus may include a use management part. The use management part may further include a storage part 13 and a communication part 18.

In one aspect, the use management part may manage use information of a user based on control content of the control part 12. In one embodiment, the use information may be content about the user usage of the hydrogen generation apparatus and may be, for example, a water electrolysis time, an amount of electrolyzed water, a resistance, a type of electrolyte, a concentration of an electrolyte, and an amount of generated hydrogen. In addition, the use information may also include information such as magnitudes of a voltage and current applied for electrolysis.

In one aspect, the storage part 13 may store the use information of a user managed by the use management part. Here, content stored in the storage part 13 may not be modified. Therefore, the user may safely manage the use information.

In one aspect, the communication part 18 may be connected to an apparatus use expert system 30 to transmit the use information thereto or receive a guide related to the use information therefrom. In one embodiment, the communication part 18 may be a wireless communication module and may communicate with a short-range communication module such as a Bluetooth of a smart phone or a tablet personal computer (PC) or may communicate with a near field communication (NFC) module. The apparatus use expert system 30 may be a system of a company that supplies the hydrogen generation apparatus or a company that specifically manages the use of the apparatus. The system may receive the use information of the hydrogen generation apparatus of a user via the communication part 18, analyze the use information, and generate a suitable user guide for the user on the basis of an analysis result. In addition, the system may transmit user guide information to the user to guide the proper use of the hydrogen generation apparatus. The hydrogen generation apparatus may receive the corresponding information via the communication part 18 and inform the user of the corresponding information.

In one aspect, the hydrogen generation apparatus includes a waterproof space. The waterproof space is separated from the electrolysis electrode part 3 and sealed such that water does not come into contact with the waterproof space. The waterproof space may include a first magnetically-sensitive power isolation switch 24. The first magnetically-sensitive power isolation switch may be operated by a magnetic force to adjust power of the hydrogen generation apparatus to be turned on and off without physical contact with a magnet.

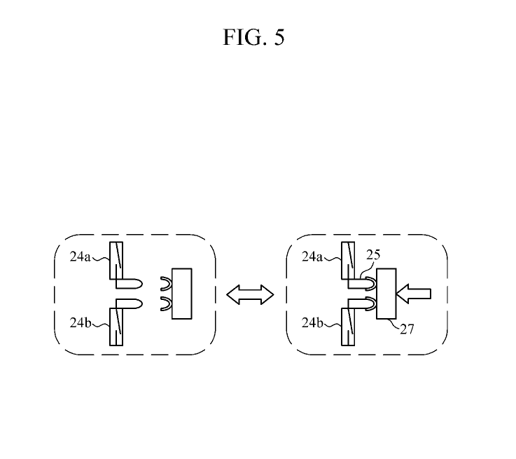

In one additional aspect, the waterproof space may further include a second magnetically-sensitive power isolation switch 24 and an exposed boosting charge part 25. The second magnetically-sensitive power isolation switch 24 is adjusted by a magnetic force without physical contact with a magnet. The exposed boosting charge part 25 protrudes outward from the waterproof space, and in a case in which a magnet comes into contact with the exposed boosting charge part 25, the exposed boosting charge part 25 is connected to the magnetically-sensitive power isolation switch to charge a secondary battery. In one embodiment, the first magnetically-sensitive power isolation switch and the second magnetically-sensitive power isolation switch are connected to a quick charge circuit of the secondary battery. When a magnet comes into contact with the exposed boosting charge part 25, the first magnetically-sensitive power isolation switch and the second magnetically-sensitive power isolation switch are switched by a magnetic force, a charging current flows in the sealed hydrogen generation apparatus. Accordingly, the charging current also flows in the quick charge circuit of the secondary battery and the secondary battery to charge the secondary battery. FIG. 5 is a view illustrating first and second magnetically-sensitive power isolation switches 24a and 24b, the exposed boosting charge part 25 and a boosting charge electrode magnet 27.

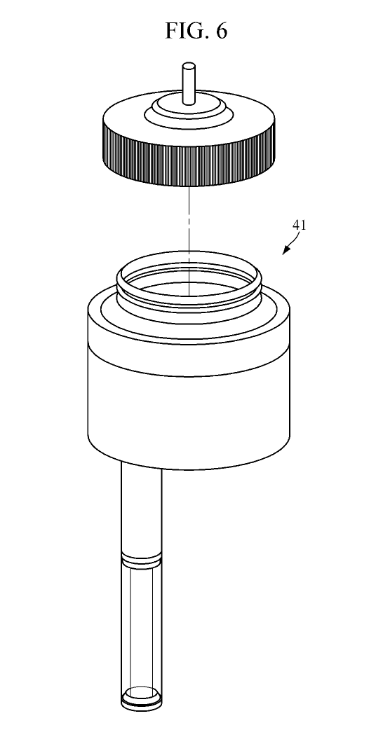

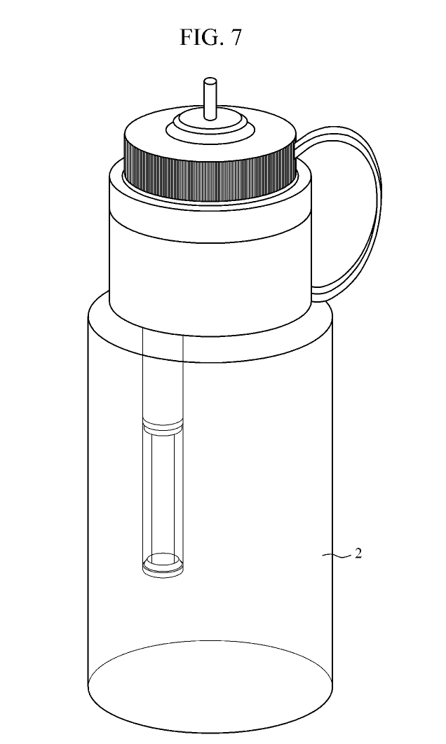

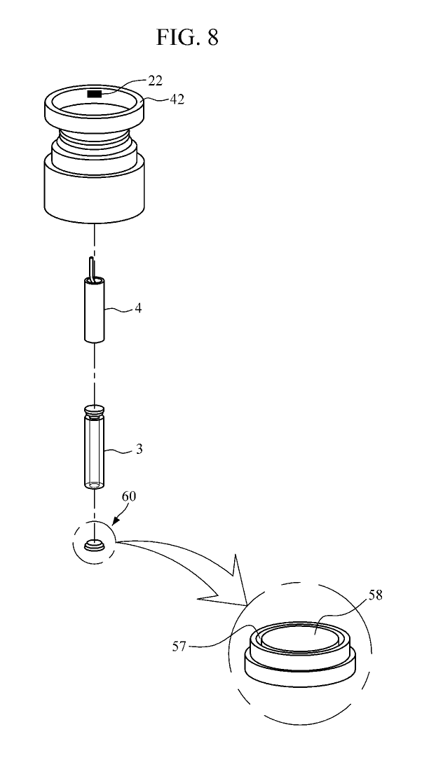

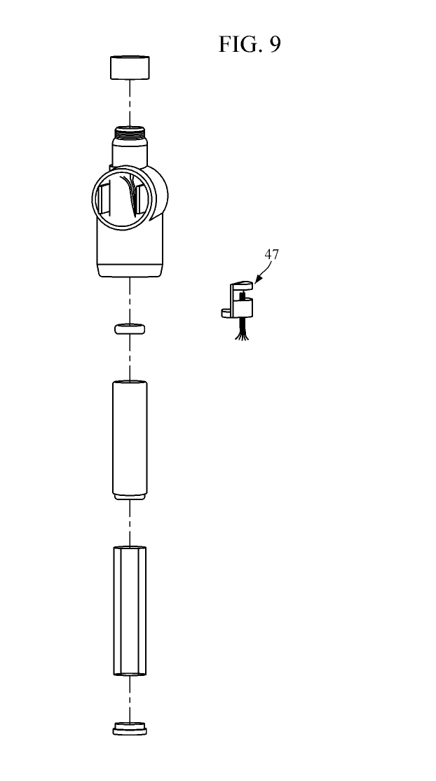

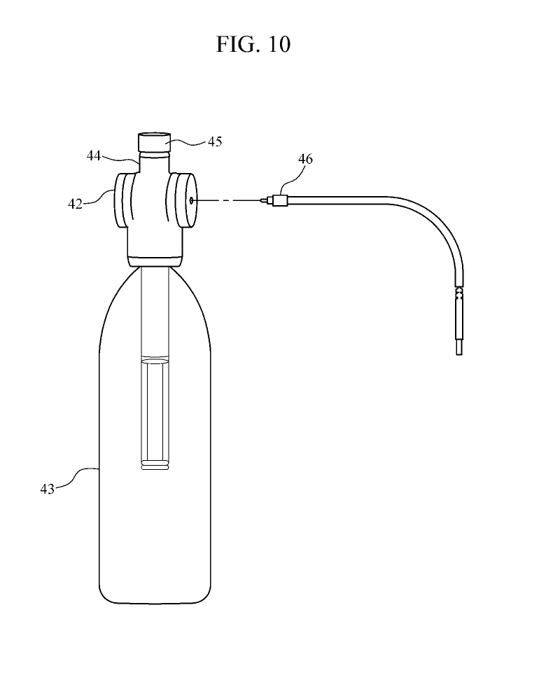

FIG. 6 is a view illustrating one example of the hydrogen generation apparatus including a circuit of FIG. 1. FIG. 7 is a view illustrating the hydrogen generation apparatus of FIG. 6 coupled to a water bottle. FIG. 8 is an exploded view illustrating the hydrogen generation apparatus of FIG. 6.

In one aspect, the hydrogen generation apparatus includes a main body 41. The main body 41 may include the waterproof space 23 therein. In a specific aspect, the main body 41 further includes a water bottle coupling portion, a rotary switch 42, an electrolysis electrode part 3, and a fixing groove.

The water bottle coupling portion includes a female thread formed at a lower end of the main body such that the main body 41 is coupled to the water bottle 2. The rotary switch 42 having a ring structure operates by rotating the magnetically-sensitive power isolation switches. For example, as the rotary switch 42 includes a magnet 22, the magnet is rotated when the rotary switch is rotated, and the magnetically-sensitive power isolation switches are operated according to movement of the magnet.

In a specific aspect, the electrolysis electrode part 3 may include the above-described electrolysis electrode and the above-described power supply part 11, and further include a protruding part. In one embodiment, the electrolysis electrode part 3 may be a rod having a cylindrical shape. FIG. 8 is a view specifically illustrating the electrolysis electrode part 3, which is a rod having a cylindrical shape.

In a specific aspect, the electrolysis electrode part 3, which is a rod having a cylindrical shape, protrudes from the lower end of the main body after the protruding part is coupled to the fixing groove and enters the water bottle when the main body is coupled to the water bottle. In one embodiment, the protruding part located at an upper end of the electrolysis electrode part 3 is inserted into the fixing groove located in the main body such that the electrolysis electrode part is coupled to the main body and the electrolysis electrode part is not shaken. After coupling, the electrolysis electrode part 3 protrudes from the lower end of the main body 41 as illustrated in FIG. 6.

In one aspect, the main body may further include a vertical path and a cap coupling portion. Water may pass through the vertical path. When the main body is coupled to the water bottle as illustrated in FIG. 5, the vertical path is located in the main body such that water in the water bottle may flow through holes located in an upper end of the main body. The cap coupling portion includes a male thread to be coupled to a cover configured to open and close the path.

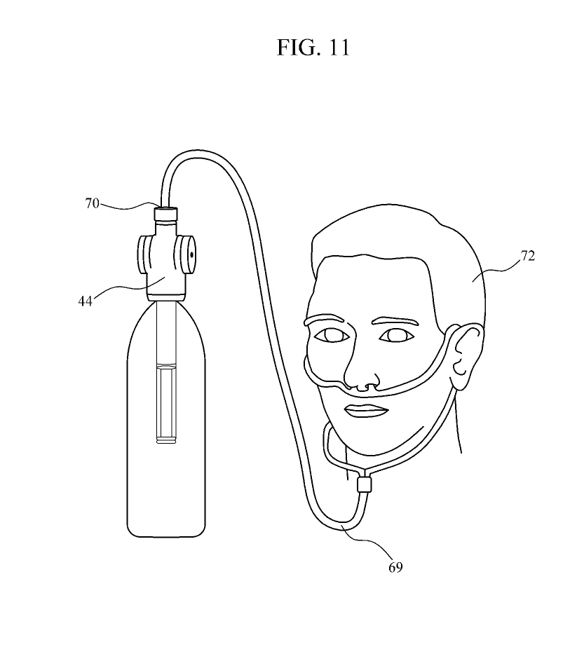

In one aspect, the main body 41 may include a nozzle. The nozzle 70 may be connected to a hose of a hydrogen respirator. Alternatively, a nozzle may be included in the cover configured to cover the main body 41. FIGS. 6, 7, and 11 are views illustrating the nozzle located at the cap and a hydrogen respirator 69 connected to the nozzle. A user 72 may inhale hydrogen gas using the hydrogen respirator 69 connected to the nozzle 70.

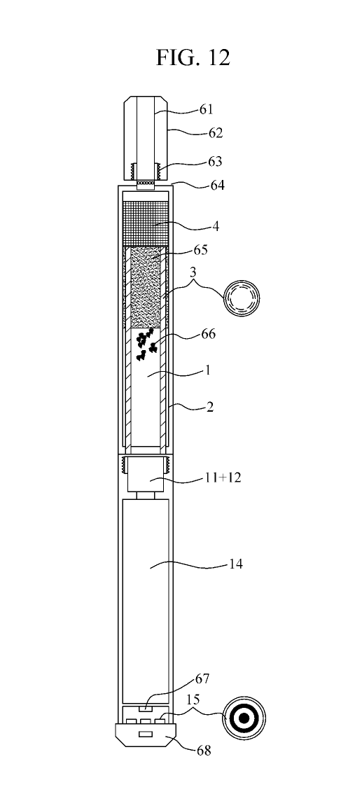

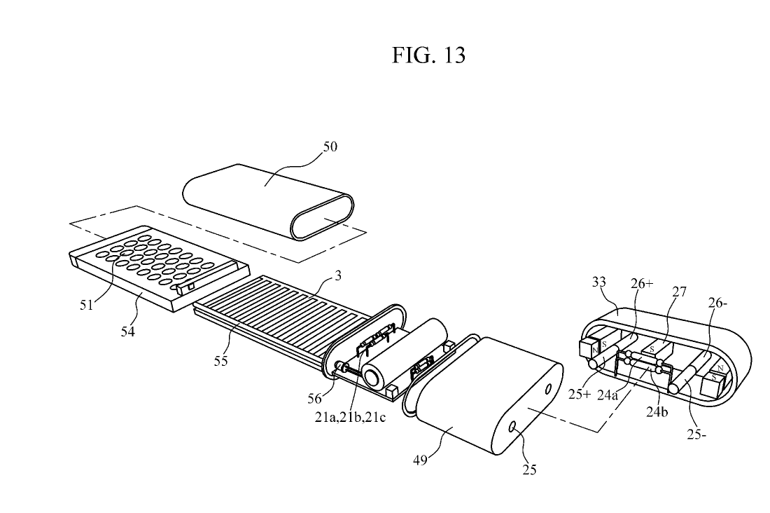

FIG. 12 is a view illustrating the hydrogen generation apparatus having an electronic cigarette form. The hydrogen generation apparatus in the electronic cigarette form includes the power supply part 11, a hydrogen generation part, and an inhalation part 61.

In one embodiment, the power supply part 11 may include a charging circuit 15, the secondary battery, and the isolated DC-DC converter.

In one aspect, the hydrogen generation part and the power supply part may be coupled at one side of the hydrogen generation apparatus and may also be separated.

In one aspect, the inhalation part 61 and the hydrogen generation part may be coupled at the other side opposite the one side at which the power supply part 11 and the hydrogen generation part are coupled.

In a specific aspect, the porous ceramic catalyst 4 configured to be combined with oxygen generated when water is electrolyzed to prevent the generation of ozone is located in the hydrogen generation part, and the water bottle 2 of the hydrogen generation apparatus includes a liquid absorbent 65 in which parts of the corresponding electrolysis electrodes 3 are buried at one side at which the electrolysis electrodes 3 in the cylindrical shape having the vertical path is coupled to the inhalation part 61. The electrolysis electrodes 3 have the cylindrical shape having a hollow center, that is, a concentric shape. Accordingly, the area of electrode part in contact with water may be increased. The liquid absorbent 65 may be a porous sponge for preventing water from leaking and may isolate water to allow only hydrogen bubbles 66 to pass through the liquid absorbent 65.

In one aspect, the inhalation part 61 may further include an inhalation part, micro holes 63, and an adjustor 62. A user may inhale hydrogen through the inhalation part, and external air, which will be mixed with the hydrogen inhaled through the corresponding inhalation part by the user during inhalation may enter the hydrogen generation apparatus through the micro holes 63. The adjustor 62 may be rotated to open and close the micro holes 63. In one embodiment, when the adjustor 62 is rotated to open the micro holes, hydrogen is mixed with air, the user may inhale the hydrogen mixed with the air when the user's mouth is in contact with the inhalation part and inhales the hydrogen.

1. A hydrogen generation apparatus configured to electrolyze water to generate hydrogen, comprising:
an electrolysis electrode part which electrolyzes the water and in which a porous ceramic catalyst combined with oxygen generated during the electrolysis to prevent generation of ozone is located;
a resistance measuring part configured to measure a resistance of the water and measure a change in the resistance based on a type of an electrolyte dissolved in the water and a concentration of the corresponding electrolyte;
a power supply part configured to apply a voltage isolated from a power device to electrolysis electrode parts;
a control part configured to control the power supply part to change and apply the voltage on the basis of the resistance measured by the resistance measuring part; and
a relay part controlled to apply the voltage to the electrolysis electrodes while changing a direction of current at a predetermined time interval.

2. The hydrogen generation apparatus of claim 1, further comprising a light-emitting diode configured to flicker or light up in a combination of red, blue, and a green to inform of operating and stopping of electrolysis,
wherein the control part controls the relay part to stop power supply to stop electrolysis in a case in which the resistance measuring part measures a resistance of the water in which a salinity is 10% or more and controls the light-emitting diode to inform of the stopping of electrolysis.

3. The hydrogen generation apparatus of claim 1, wherein the control part controls the relay part to decrease the applied voltage in a case in which the resistance measuring part measures a resistance of the water in which a salinity is equal to or higher than 4% and lower than 10%, and to electrolyze the water for an added voltage application time period corresponding to the lowered voltage.

4. The hydrogen generation apparatus of claim 1, wherein the power supply part and the relay part are controlled to electrolyze the water at a maximum voltage for a predetermined time period in a case in which the resistance measuring part measures a resistance of water in which a salinity is lower than 3%.

5. The hydrogen generation apparatus of claim 1, wherein:
the resistance measuring part measures a change amount of the resistance based on an amount of water; and
the control part controls the relay part to supply power on the basis of the change amount of the measured resistance to change a time for which water is electrolyzed.

6. The hydrogen generation apparatus of claim 1, wherein the control part controls the power supply part to apply a voltage lower than an initial voltage isolated from a power supply voltage in a case in which a resistance between both ends of the electrolysis electrodes is not measured and the initial voltage is applied to the both ends of the electrodes and excessive current flows at the both ends of the electrode.

7. The hydrogen generation apparatus of claim 1, further comprising a gyro sensor configured to detect an inclination of a water bottle,
wherein the control part controls the power supply part to stop power supply in a case in which an inclination of the water bottle detected by the gyro sensor is greater than a predetermined angle.

8. The hydrogen generation apparatus of claim 1, further comprising a water level detecting electrode configured to detect contact with water,
wherein the control part controls the power supply part to stop power supply in a case in which the water level detecting electrode detects contact with water.

9. The hydrogen generation apparatus of claim 1, further comprising a pressure sensor configured to detect a pressure of the hydrogen generated by the water being electrolyzed,
wherein the control part controls the power supply part to stop power supply in a case in which a pressure detected by the pressure sensor is higher than a predetermined pressure.

10. The hydrogen generation apparatus of claim 1, further comprising a use management part configured to manage use information of a user based on control content of the control part,
wherein the use management part further includes:
a storage part which stores the use information and in which modification is impossible after the use information is stored; and
a communication part connected to an apparatus use expert system and configured to transmit the use information to the apparatus use expert system, or receive a guide about the use information from the apparatus use expert system.

11. The hydrogen generation apparatus of claim 1, further comprising a waterproof space including a first magnetically-sensitive power isolation switch operated by a magnetic force without physical contact with a magnet to adjust power of the hydrogen generation apparatus to be turned on and off, separated from the electrolysis electrode part, and sealed to prevent contact with water.

12. The hydrogen generation apparatus of claim 1, wherein the waterproof space further includes:
a second magnetically-sensitive power isolation switch adjusted by a magnetic force without physical contact with a magnet; and
an exposed boosting charge part including an exposed boosting charge electrode configured to protrude outward from the waterproof space, and connected to the magnetically-sensitive power isolation switch to charge a secondary battery in a case in which the exposed boosting charge electrode comes into contact with a magnet.

13. The hydrogen generation apparatus of claim 11, wherein:
the waterproof space is included in a main body;
the main body includes a water bottle coupling portion including a female thread formed at a lower end of the main body to couple the main body to a water bottle, a rotary switch having a ring structure and which operates by rotating the first magnetically-sensitive power isolation switch, an electrolysis electrode part formed in a cylindrical shape and including the electrolysis electrode part, the power supply part, and a protruding part formed at an upper end of the electrolysis electrode part, and a fixing groove into which the protruding part is inserted to couple the electrolysis electrode part to the main body; and
the electrolysis electrode part protrudes downward from the lower end of the main body after the protruding part is coupled to the fixing groove and enters the water bottle when the main body is coupled to the water bottle.

14. The hydrogen generation apparatus of claim 13, wherein the main body further includes:
a vertical path through which water passes; and
a cap coupling portion including a male thread formed at an upper end of the cap coupling portion to be coupled to a cover configured to open and close the path.

15. The hydrogen generation apparatus of claim 14, wherein the main body or the cover further includes a nozzle connected to a hose of a hydrogen respirator.

16. A hydrogen generation apparatus having an electronic cigarette form, comprising:
a power supply part including a charge circuit, a secondary battery, and an isolated DC-DC converter;
a hydrogen generation part coupled to the power supply part at one side and configured to be separable from the power supply part; and
an inhalation part coupled to the hydrogen generation part at the other side opposite the one side,
wherein:
a porous ceramic catalyst configured to be combined with oxygen generated when water is electrolyzed to prevent ozone generation is located in the hydrogen generation part; and
a water bottle includes a liquid absorbent in which parts of electrolysis electrodes are buried at one side at which the electrolysis electrodes in a cylindrical shape having a vertical path are coupled to the inhalation part.

17. The hydrogen generation apparatus of claim 16, wherein the inhalation part includes:
an inhalation part through which a user inhales hydrogen;
a micro hole through which external air to be mixed with the hydrogen enters when the user inhales the hydrogen using the inhalation part; and
an adjustor configured to open and close the micro hole by rotating.
